(19) United States
(12) Patent Application Publication
(10) Pub. No.: US 2011/0104773 A1
(43) Pub. Date: May 5, 2011

(54) PROCESSING METHOD FOR FRACTIONALLY CONVERTING PENNISETUM HYDRIDUM INTO FUEL ETHANOL WITH CO-PRODUCTION OF ELECTRICITY GENERATION AND PAPER PULP

(75) Inventors: Hongzhang Chen, Beijing (CN); Jianxing Zhang, Beijing (CN); Cheuk Lam Leung, Hongking (CN); Yong Li, Beijing (CN)

(73) Assignee: Green Resources Technology Limited, Hong Kong (CN)

(21) Appl. No.: 12/312,201

(22) PCT Filed: Feb. 15, 2008
(86) PCT No.: PCT/CN2008/001474

§ 371 Date: Jan. 19, 2011

(30) Foreign Application Priority Data
Feb. 27, 2008 (CN) 200810100967.1

(51) Int. Cl. C12P 7/06

(52) U.S. Cl. 435/161

(57) ABSTRACT

The invention relates to a method for fractionally converting pennisetum hydridum into fuel ethanol with co-production of electricity generation and paper pulp, which comprises subjecting the pennisetum hydridum having been cut into segments to steam explosion treatment by utilizing a steam explosion device to obtain a steam explosion product, and said method is characterized in that it comprises the steps of: washing the steam explosion product with water to obtain fibers and water washing liquid containing a degraded hemicellulose, and separating and purifying the water washing liquid to obtain an xylo-oligosaccharide; fractionally teasing the fibers, and subjecting the obtained long fibers to an ethanol self-catalyzed pulping and then a bleaching treatment to obtain a paper pulp, and after recovering ethanol, obtaining an alcohol-soluble lignin by separation; preparing cellulase by a solid state fermentation, that is, taking the short fibers obtained by fractionally teasing as a fermentation substrate of Trichoderma viride to prepare a solid cellulase; mixing the short fibers, the solid cellulase and yeast to perform a simultaneous saccharification fermentation for obtaining the fuel ethanol and a fermentation residue, and producing CO₂ by the fermentation; and drying the fermentation residue to prepare a biomass fuel for electricity generation. The method of the invention can realize a high efficient comprehensive utilization of the pennisetum hydridum resource.

### FIELD OF THE INVENTION

The present invention relates to the comprehensive utilization of pennisetum hydridum resource, and specifically, to a method for producing fuel ethanol, paper pulp and a biomass fuel for electricity generation by utilizing pennisetum hydridum.

### BACKGROUND

With the swift and violent increase of the population in the world and the continuous development of the social economy, in recent 400 years, the human almost exhausted the fossil energy sources of petroleum, coal and natural gas accumulated in the past 2.5 billion years on the earth. Since China became a net import country for petroleum products for the first time in 1993, the crude oil import amount of China has exceeded 100 million tons in 2004, and therefore, in order to solve the problem of national energy security, it is emergent to find clean, cheap and reproducible energy source substitutes.

In the energy sources which may substitute the fossil energy sources, biomass has drawn much attention of the world due to its advantages of reproducibility, great output, storability, carbon circulation and the like. Additionally, the biomass energy source is an exclusive reproducible energy source which can be converted into fuel ethanol. The fuel ethanol has great compatibility with the liquid fossil energy sources such as gasoline in the manner of utilization. In the industrial scale of the biomass energy products, the fuel ethanol develops fastest. At present, in Brazil, there are more than 300 sugarcane processing factories and the fuel ethanol output thereof is 17 billion liters per year; in USA, 10 million tons of fuel ethanol was produced by taking corn as raw material in 2004. At present, the total ethanol producing capacity of the four main companies in China, that is, Fengyuan Biochemical (Anhui Province), Huarun Alcohol (Heilongjiang Province), Tianguan (Henan Province) and Alcohol (Jilin Province), is 1.63 million tons per year, and the total output thereof is about 1.4 million tons. However, in China, the fuel ethanol is mostly converted from corn nowadays, which is high in raw material cost and is in competition with the human for foodstuff. On the other hand, a fermentation conversion utilizing lignocellulose raw material accords with the principle of “no competition with human for foodstuff and no competition with to foodstuff for soil” and has an attractive perspective for producing fuel ethanol. The advantage of producing fuel ethanol by fermenting the lignocellulose raw material is obvious, and furthermore, pennisetum hydridum has been recognized gradually due to its advantages of high biomass accumulation, high thermal value, water and soil maintainability and the like.

Pennisetum hydridum is a high quality fodder grass with high output and high protein which was introduced from Colombia, South America into China. It is referred to as “king of grass” and has the aliases of king grass, emperor bamboo and giant elephant grass, as well as a Latin name of Pennisetum purpureum Schum. It belongs to angiosperm phylum, monocotyledoneae class, gramineae family and pennisetum genus. Pennisetum hydridum is a high yielding, high quality gramineae fodder grass cultivated by hybridizing elephant grass with American pennisetum. The pennisetum hydridum growing straightly in a manner of cluster has tall plant, advanced root system, and is a perennial plant in the areas with adequate temperature. The plant has a height up to 4-5 meters and a length of 9-15 cm between joints; there are 15-30 effective sprouts, and one axillary bud is grown on each joint and is encapsulated with leaves with a length of 60-132 cm and a width of 3-6 cm growing superposedly. The planting of pennisetum hydridum in the areas of Guangdong Province, Guangxi Province and the like in China has the advantages of fast effect, long harvest period, as well as stable and high output. Pennisetum hydridum can be reaped in 2-3 months after being planted in spring and will keep growing with the reaping. It can be reaped for 4-6 times and can be reaped for 6-7 years for once planting. The output per year for each 1/15 hectare is up to 25 tons.

The main chemical ingredients of pennisetum hydridum include cellulose, hemi-cellulose and lignin, and these three ingredients constitute the supporting skeleton of the plant body; wherein, the cellulose forms microfibers which constitute the netlike skeleton of the cell walls of the plant, while the hemi-cellulose and lignin are the “adhesive” and “filler” filled between the fibers. In pennisetum hydridum, the total content of cellulose is 75% or more, the content of Classen lignin is 20% or more, and ash comprises about 3%. Additionally, the pennisetum hydridum further comprises pectin substances, lipids and waxes, lipins, low-molecular carbohydrates and the like.

At present, the main application of pennisetum hydridum in industry is being a fine quality fodder grass and a new raw material for feedstuff, papermaking and construction material. \textcircled{1} As raw material for fodder grass: pennisetum hydridum is the best succulence for feeding phytophagous livestocks, fowls and fishes. It has a large harvest amount per year and can be reaped for a long period. However, when pennisetum hydridum is used as feedstuff directly, it has the disadvantages of difficult digesting absorption, poor palatability and low added value. Chinese Invention Patents 200410040941.4, 200610138365.6 have respectively introduced the methods for producing protein feedstuffs by utilizing pennisetum hydridum. \textcircled{2} As raw material for papermaking: pennisetum hydridum is a fast-growing paper-making raw material with great potential and good papermaking performance. The pennisetum hydridum has the advantages of long fiber length, high aspect ratio, good beatability, soft fibers, relatively high tearing strength and the like. It is suitable for preparing top grade paper pulp and dissolved pulp, and can be used as a raw material for papermaking instead of wood so as to save wood for the country. A method for preparing bleached pulp by utilizing pennisetum hydridum as raw material was introduced by Hui Zhang from Nanjing Forestry University. \textcircled{3} As raw material for building material: Chinese Invention Patents 200410016746.8, 200610011006.4 introduced respectively that, the building material boards of fiber boards, medium-density boards, molded board and the like with excellent quality and low price, as well as various artworks, can be manufactured by utilizing the plant of pennisetum hydridum.

However, the above applications of pennisetum hydridum are only aimed at the utilization of a single component in pennisetum hydridum or the utilization with low added value. For example, as a papermaking raw material, only the cellulose in pennisetum hydridum is utilized and the other components of hemi-cellulose, lignin and the like are discharged as papermaking waste liquid, and therefore, not only the resource is wasted, but also the environment is polluted seriously. Thus, in view of the integrated aspects of how to increase the added value of pennisetum hydridum, how to save the resource or the like, it is imperative under the situation to develop a processing route for the comprehensive utilization of pennisetum hydridum. Planting and managing pennisetum hydridum being an energy source crop will provide a new route for solving the energy source problem in China.

Additionally, in recent years, a technology of biomass electricity generation develops rapidly. In the countries of Austria, Denmark, Finland, France, Norway, Sweden, America and the like, the biomass has drawn more and more attentions for being used as a fuel for electricity generation. In China, since 1987, a research work on the biomass energy miniaturized gasification electricity generation technology has been carried out and was regarded as a key project of the Ministry of Science and Technology. In 2000, a demonstrating project for straw gasification electricity generation was performed in China. However, because the reasons of low thermal value, small density, relative dispersedness and the like for the biomass, the electricity generation by utilizing biomass can not make a breakthrough in economical cost. Therefore, in order to satisfy the national energy source demand and increase the comprehensive utilization value of pennisetum hydridum, it is further needed to carry out an in-depth research on producing fuel ethanol with co-production of a biomass fuel for electricity generation by fermenting pennisetum hydridum.

### DISCLOSURE OF THE INVENTION

A main object of the invention is to provide a method for producing fuel ethanol, xylo-oligosaccharide, paper pulp, alcohol-soluble lignin and a biomass to fuel for electricity generation by utilizing pennisetum hydridum as raw material.

The technical solutions of the invention are as follows:

(1) A method for fractionally converting pennisetum hydridum into fuel ethanol with co-production of electricity generation and paper pulp is provided, which comprises taking pennisetum hydridum as raw material and subjecting the pennisetum hydridum cut into segments to steam explosion treatment by utilizing a steam explosion device to obtain a steam explosion product, and said method is characterized in comprising the steps of:
water washing: washing the steam explosion product with water to obtain fibers and water washing liquid containing degraded hemi-cellulose, and separating and purifying the water washing liquid to obtain xylo-oligosaccharide;
fiber fractionation: fractionally teasing the fibers to obtain long fibers and short fibers;
pulping: subjecting the long fibers to an ethanol self-catalyzed pulping and then a bleaching treatment to obtain paper pulp and alcohol-soluble lignin;
preparing cellulase by solid state fermentation: taking the short fibers as fermentation substrate of Trichoderma viride to prepare cellulase;
simultaneous saccharification fermentation: subjecting the short fibers and the fermentation cellulase to simultaneous saccharification fermentation to obtain fuel ethanol and fermentation residue;
drying the fermentation residue: drying the fermentation residue to prepare biomass fuel for electricity generation.

(2) The method according to item (1), characterized in that in the step of steam explosion, the steam pressure is 1.5 MPa.

(3) The method according to item (2), further characterized in that in the step of steam explosion, the steam pressure is maintained for 3-5 minutes.

(4) The method according to item (1), characterized in that the step of the simultaneous saccharification fermentation further comprises recovering CO₂ produced by the fermentation through a water-draining and gas-collecting method.

(5) The method according to item (1), characterized in that in the step of the pulping, the ethanol self-catalyzed pulping comprises the steps of:
cooking: subjecting the long fibers and an aqueous solution of 60% ethanol by volume to pulping in a high pressure reaction tank at 160° C. for 2 h;
separation: after the cooking is completed, the reaction system is cooled to room temperature and filtered to obtain filtrate and crude slurry, and after distilling the obtained filtrate to recover ethanol, the alcohol-soluble lignin is obtained;
bleaching: subjecting the crude slurry to bleaching treatment to obtain the paper pulp.

The long fibers and the short fibers described in the invention have the lengths defined as follows: a fiber with a fiber length of 3 cm or more is defined as the long fiber, and a fiber with a fiber length less than 3 cm is defined as the short fiber.

In the step of preparing cellulase by solid state fermentation in the method of the invention, Trichoderma viride 3.1044, is purchased from The Microbiological Culture Center of Institute of Biophysics, Chinese Academy of Sciences, Beijing, China.

The method of the invention is a process for full utilization of the components in pennisetum hydridum, which obtains an object product of fuel ethanol, and simultaneously, co-produces biomass fuel (fermentation residual) for electricity generation, xylo-oligosaccharide, paper pulp and alcohol-soluble lignin, and recovers CO₂ produced by the fermentation. The method of the invention relates to a green environmental and high value comprehensive utilization of pennisetum hydridum, and overcomes the traditional disadvantage of product simplification in utilizing pennisetum hydridum resources.

The steam exploded long fibers obtained by the method of the invention have high strength and good toughness, which is suitable for producing medium and top grade paper pulp; a part of the steam exploded short fibers obtained by separation firstly performs solid state fermentation for preparing cellulase, and the obtained cellulase performs simultaneous saccharification fermentation with the other part of steam exploded short fibers for preparing ethanol; after the fermentation, the residue after a dehydration treatment may be used for biomass electricity generation and CO₂ produced during the fermentation is recovered. By the processing flow, not only fuel ethanol is obtained, but also fermentation residue electricity generation, xylo-oligosaccharide, paper pulp, alcohol-soluble lignin and CO₂ produced by the fermentation are co-produced, which makes the added value of pennisetum hydridum increase obviously.

There are many researches on pulping and papermaking by utilizing pennisetum hydridum, however, the present process provides a utilization technology of fractional conversion for co-producing paper pulp by aiming at the particularity of pennisetum hydridum, rather than pulping directly by utilizing the whole plantlet of pennisetum hydridum. Additionally, the process performs pulping using the ethanol self-catalysis to co-produce alcohol-soluble lignin. The obtained lignin can be used as dispersant, adsorbent/desorbing agent, petroleum recovering aid, asphalt emulsifier and the like, and has a very wide application perspective.

The fermentation residue of pennisetum hydridum during the production can be used as biomass fuel for electricity generation. In the fermentation residue of pennisetum hydridum after steam explosion and ethanol production by microorganism fermentation, the ratio of lignin increases obviously and the content of oxygen element drops. Because the thermal value of lignin is higher than that of hemi-cellulose and cellulose, the thermal value of the fermentation residue after the fermentation may increase. Furthermore, in the residue after the treatment of steam explosion and microorganism fermentation, the element contents of chlorine and sulphur decrease obviously and the melting point of ash increases. These properties are of great benefit to the biomass electricity generation.

In summary, the invention has the advantageous effects as follows:

\textcircled{1} According to the characteristic of compact structure for pennisetum hydridum, pennisetum hydridum is subjected to a pre-treatment of steam explosion which increases the fiber dispersion degree of the pennisetum hydridum greatly and benefits the growth of microorganism and the separation of the long and short fibers;

\textcircled{2} Aiming at the previous disadvantage of a single product for pennisetum hydridum, a full utilization technology for pennisetum hydridum is emphasized, which increases the utilization ratio of pennisetum hydridum and saves the resource greatly;

\textcircled{3} By producing fuel ethanol by pennisetum hydridum fermentation with co-production of products with high added value of biomass electricity generation, xylo-oligosaccharide, paper pulp, alcohol-soluble lignin, recovered CO₂ and the like, the comprehensive utilization route for pennisetum hydridum is optimized;

\textcircled{4} By overcoming the disadvantages of the pollution of the traditional methods for component separation and single component utilization, a clean full utilization of pennisetum hydridum is realized.

### DESCRIPTION OF THE DRAWING

FIG. 1 is a flow diagram of the method of the invention.

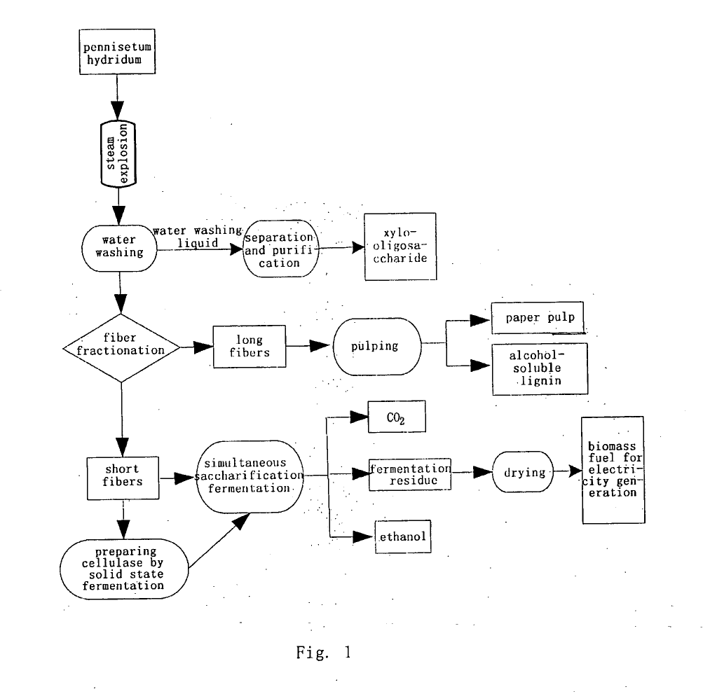

### MODE FOR CARRYING OUT THE INVENTION

### Example 1

\textcircled{1} Steam explosion treatment: performing a segment cutting treatment of pennisetum hydridum, and subjecting the pennisetum hydridum cut into segments (with a cutting length of 3-5 cm) to a steam explosion treatment utilizing a steam explosion device developed independently by Institute of Process Engineering, Chinese Academy of Sciences (see Chinese Invention Patent ZL01218677.5: Double-way Air Inlet Quick Door Open Steam Explosion Reaction Tank) under a pressure of 1.5 MPa for 3 min.

\textcircled{2} The preparation of xylo-oligosaccharide: the pennisetum hydridum after steam explosion was treated with a two-stage counter-current water washing (the water washing liquid of the first stage was used for washing the steam exploded pennisetum hydridum of the second stage) for 4 h to obtain fibers and water washing liquid containing hemi-cellulose. The water washing liquid was filtered to remove solid impurities therein and was subjected to vacuum rotating evaporation at 75° C. to be concentrated to be ¼ of the original volume thereof. Then, the concentrated liquid was decolorized with active carbon for 24 h to remove the excess monosaccharides. Subsequently, the concentrated liquid was separated and purified through an active carbon chromography column by 60% ethanol eluting. The collected eluent was assayed with 3,5-dinitrosalicylic acid and the collection was stopped until the eluent contained no reducing sugar any more. The eluent in the tube of the reducing sugar was mixed and taken as an active xylo-oligosaccharide. Under a temperature of 40° C., the collected active xylo-oligosaccharide was subjected to reduced pressure evaporation to remove the ethanol contained therein (which can be recovered for repeated application), and then to nano-filtration to obtain sirup containing 50% active xylo-oligosaccharide.

\textcircled{3} Fractionation of the long and short fibers: after subjecting 5000 g of the fibers after the water washing to a fractional teasing using a mechanical teasing device (Paul screener, ZT10-00, Xingping Zhongtong Experimental Equipment Co., Ltd.), 3570 g of long fibers (with a fiber length more than 3 cm) and 1430 g of short fibers (with a fiber length less than 3 cm) were obtained.

\textcircled{4} The long fiber pulping: 100 g of the teased long fibers were maintained in a high pressure reaction tank (GCF-1.0 L high pressure reaction tank, Dalian self-control Equipment Factory) for 2 h at 160° C. with a ethanol to concentration of 60% and a solid-liquid ratio of 1/10 (g/ml) to remove lignin. After the cooking was completed, crude slurry was obtained. The reaction system was cooled to room temperature and filtered to obtain filtrate and crude slurry.

Because the lignin was insoluble in water, the obtained filtrate was evaporated with a vacuum rotating evaporator at 60° C. to recover ethanol, and the lignin was separated. After filtering with a Buchner funner, the filter residue was obtained as the alcohol-soluble lignin.

Then, the crude slurry was subjected to bleaching utilizing a two-stage bleaching technology in which each stage was performed for 2 h under 70° C., wherein, the composition of the bleaching liquid for the first stage was 1% Na₂SiO₃, 2% H₂O₂, 2% NaOH, the composition of the bleaching liquid for the second stage was 1% Na₂SiO₃, 1% H₂O₂, 1% NaOH and the solid-liquid ratio of both stages were 1/20 (v/v). After drying the bleached pulp, a paper pulp was obtained and weighted for calculating the yield of the paper pulp.

\textcircled{5} Preparing cellulase by the solid state fermentation of the short fibers: 4 g of the teased short fibers and 1 g of wheat bran were wetted by 12 ml inorganic salt solution (1.5 g of (NH₄)₂SO₄, 0.6 g of MgSO₄ and 0.3 g of KH₂PO₄ were contained per 100 ml solution) and mixed uniformly. After being sterilized at 121° C. for 30 min, the mixture was added with 3 ml of seeding solution of trichoderma viride 3.1044, and cultured at 30° C. for 4 days. Then, the weight loss and Filter Paper Enzyme Acticity (FPA) of the sample were measured.

\textcircled{6} Simultaneous saccharification fermentation of the short fibers: with a solid-liquid ratio of 1/20 (v/v), each grain of the substrate was added with 20 IU FPA cellulase and 0.01 g yeast (produced by Angel Yeast Co., Ltd., China) and was fermented at 37° C. for 72 h. During the fermentation, the produced CO₂ was recovered by a water-draining and gas-collecting method.

\textcircled{7} The fermentation residue after the simultaneous saccharification was dried at 80° C. to obtain biomass fuel for electricity generation.

The yield indices of the obtained product are as follows:

The removal percentage of hemi-cellulose at water washing the steam explosion product: 56.3%;

The yield of xylo-oligosaccharide: 0.0041 g xylo-oligosaccharide/g pennisetum hydridum;

The ratio of the long fibers and the short fibers: 1:0.4;

The yields of crude pulp and paper pulp, as well as the lignin removing percentage at the long fiber pulping were 54.3%, 39.5% and 72.6%, respectibely;

The yield of alcohol-soluble lignin: 7.36%;

The weight loss and Filter Paper Enzyme Acticity (FPA) of the cellulase sample after solid state fermentation were 23.2% and 30.4 (IU/g dried raw starter) respectively;

The ethanol yield of the simultaneous saccharification fermentation: 0.108 g ethanol/g substrate;

The yield of CO₂: 0.45 g CO₂/g ethanol;

The thermal value of pennisetum hydridum raw material (gross calorific value-air dried basis): 18.35 MJ/kg;

The thermal value of pennisetum hydridum fermentation residue (gross calorific value-air dried basis): 18.57 MJ/kg.

### Example 2

\textcircled{1} Steam explosion treatment: the method and equipment used for the steam explosion pre-treatment of pennisetum hydridum were the same as those in Example 1, and the steam explosion condition utilized was maintaining at a pressure of 1.5 MPa for 4 min.

\textcircled{2} The preparation of xylo-oligosaccharide was the same as that in Example 1.

\textcircled{3} Fractionation of the long and short fibers: after subjecting 5000 g of the fibers after the water washing to a fractional teasing using the mechanical teasing device as described in Example 1, 3425 g of long fibers (with a fiber length more than 3 cm) and 1575 g of short fibers (with a fiber length less than 3 cm) were obtained.

\textcircled{4} The long fiber pulping: the same as that in Example 1.

\textcircled{5} Preparing cellulase by the solid state fermentation of the short fibers: the same as that in Example 1.

\textcircled{6} Simultaneous saccharification fermentation of the short fibers: the same as that in Example 1.

\textcircled{7} The fermentation residue after the simultaneous saccharification was dried at 80° C. to obtain biomass fuel for electricity generation.

The obtained yield indices were as follows:

The removal percentage of hemi-cellulose at water washing the steam explosion product: 59.4%;

The yield of xylo-oligosaccharide: 0.0045 g xylo-oligosaccharide/g pennisetum hydridum;

The ratio of the long fibers and the short fibers: 1:0.46;

The yields of crude pulp and paper pulp, as well as the lignin removing percentage at the long fiber pulping were 51.8%, 36.5% and 78.2%, respectively;

The yield of alcohol-soluble lignin: 7.69%;

The weight loss and Filter Paper Enzyme Acticity (FPA) of the cellulase sample after solid state fermentation were 25.6% and 36.7 (IU/g dried raw starter) respectively;

The ethanol yield of the simultaneous saccharification fermentation: 0.116 g ethanol/g substrate;

The yield of CO₂: 0.35 g CO₂/g ethanol;

The thermal value of pennisetum hydridum raw material (gross calorific value-air dried basis): 18.35 MJ/kg;

The thermal value of pennisetum hydridum fermentation residue (gross calorific value-air dried basis): 18.72 MJ/kg.

### Example 3

\textcircled{1} Steam explosion treatment: the method and equipment used for the steam explosion pre-treatment of pennisetum hydridum were the same as those in example 1, and the steam explosion condition utilized was maintaining at a is pressure of 1.5 MPa for 5 min.

\textcircled{2} The preparation of xylo-oligosaccharide was the same as that in example 1.

\textcircled{3} Fractionation of the long and short fibers: after subjecting 5000 g of the fibers after the water washing to a fractional teasing using the mechanical teasing device as described in Example 1, 3330 g of long fibers (with a fiber length more than 3 cm) and 1660 g of short fibers (with a fiber length less than 3 cm) were obtained.

\textcircled{4} The long fiber pulping: the same as that in Example 1.

\textcircled{5} Preparing cellulase by the solid state fermentation of the short fibers: the same as that in Example 1.

\textcircled{6} Simultaneous saccharification fermentation of the short fibers: the same as that in Example 1.

\textcircled{7} The fermentation residue after the simultaneous saccharification was dried at 80° C. to obtain biomass fuel for electricity generation.

The obtained yield indices were as follows:

The removal percentage of hemi-cellulose at water washing the steam explosion product: 63.4%;

The yield of xylo-oligosaccharide: 0.0049 g xylo-oligosaccharide/g pennisetum hydridum;

The ratio of the long fibers and the short fibers: 1:0.5;

The yields of crude pulp and paper pulp, as well as the lignin removing percentage at the long fiber pulping were 52.6%, 34.5% and 83.2%, respectibely;

The yield of alcohol-soluble lignin: 7.93%;

The weight loss and Filter Paper Enzyme Acticity (FPA) of the cellulase sample after solid state fermentation were 24.6% and 39.8 (IU/g dried raw starter) respectively;

The ethanol yield of the simultaneous saccharification fermentation: 0.129 g ethanol/g substrate;

The yield of CO₂: 0.51 g CO₂/g ethanol;

The thermal value of pennisetum hydridum raw material (gross calorific value-air dried basis): 18.35 MJ/kg;

The thermal value of pennisetum hydridum fermentation residue (gross calorific value-air dried basis): 19.03 MJ/kg.

We claim:

1. A method for fractionally converting pennisetum hydridum into fuel ethanol with co-production of electricity generation and paper pulp, which comprises taking pennisetum hydridum as raw material and subjecting the pennisetum hydridum cut into segments to steam explosion treatment utilizing a steam explosion device to obtain a steam explosion product, and said method is characterized in comprising the steps of:
water washing: washing the steam explosion product with water to obtain fibers and water washing liquid containing degraded hemi-cellulose, and separating and purifying the water washing liquid to obtain xylo-oligosaccharide;
fiber fractionation: fractionally teasing the fibers to obtain long fibers and short fibers;
pulping: subjecting the long fibers to an ethanol self-catalyzed pulping and then a bleaching treatment to obtain paper pulp and alcohol-soluble lignin;
preparing cellulase by solid state fermentation: taking the short fibers as fermentation substrate of Trichoderma viride to prepare cellulase;
simultaneous saccharification fermentation: subjecting the short fibers, the fermentation cellulase and yeasts to simultaneous saccharification fermentation to obtain fuel ethanol and fermentation residue;
drying the fermentation residue: drying the fermentation residue to prepare biomass fuel for electricity generation.

2. The method according to claim 1, characterized in that in the step of the steam explosion, the steam pressure is 1.5 MPa.

3. The method according to claim 2, further characterized in that in the step of the steam explosion, the steam pressure is maintained for 3-5 minutes.

4. The method according to claim 1, characterized in that the step of the simultaneous saccharification fermentation further comprises recovering CO₂ produced by the fermentation through a water-draining and gas-collecting method.

5. The method according to claim 1, characterized in that in the step of the pulping, the ethanol self-catalyzed pulping comprises the steps of:
cooking: subjecting the long fibers and an aqueous solution of 60% ethanol by volume to pulping in a high pressure reaction tank at 160° C. for 2 h;
separation: after the cooking is completed, the reaction system is cooled to room temperature and filtered to obtain filtrate and crude slurry, and after distilling the obtained filtrate to recover ethanol, the alcohol-soluble lignin is obtained;
bleaching: subjecting the crude slurry to bleaching treatment to obtain the paper pulp.
